(19) United States
(12) Patent Application Publication
(10) Pub. No.: US 2024/0209440 A1
(43) Pub. Date: Jun. 27, 2024

(54) MULTI-SURFACE BIOLOGICAL SAMPLE IMAGING SYSTEM AND METHOD

(71) Applicant: ILLUMINA, INC., San Diego, CA (US)

(72) Inventors: Shaoping LU, Palo Alto, CA (US); Marco Krumbuegel, San Ramon, CA (US); Stanley Hong, Palo Alto, CA (US); Georg Nadorff, Rochester, NY (US)

(21) Appl. No.: 18/389,877

(22) Filed: Dec. 20, 2023

Related U.S. Application Data
(60) Provisional application No. 63/434,727, filed on Dec. 22, 2022.

(51) Int. Cl. C12Q 1/6874; B01L 3/00

(52) U.S. Cl. CPC C12Q 1/6874; B01L 3/502761; B01L 2200/0647; B01L 2200/16; B01L 2300/0654

(57) ABSTRACT

Biological samples on multiple surfaces of a support structure may be imaged using a machine comprising a lens, a flow cell and a controller. Such a machine may capture light emitted from nucleic acids disposed on first and second surfaces of the flow cell when the lens is, respectively, at first and second distances from the flow cell. In such a machine, the lens may be immersed in a first fluid, and the first and second surfaces of the flow cell may be separated by a second fluid. Additionally, in such a machine, differences between marginal and axial light rays in the field of view of the lens may be substantially equal when the lens is at the first and second distances from the flow cell.

### BACKGROUND

The disclosed technology relates generally to the field of imaging and evaluating analytical samples. More particularly, the disclosed technology relates to techniques for matching aberrations associated with imaging different surfaces of a multi-surface support structure.

There are an increasing number of applications for imaging of analytical samples on a support structure. These support structures may include plates upon which biological samples are present. For instance, these plates may include deoxyribonucleic acid (DNA) and ribonucleic acid (RNA) probes that are specific for nucleotide sequences present in genes in humans and other organisms. Individual DNA or RNA probes can be attached at specific locations in a small geometric grid or array on the support structure. Depending upon the technology employed, the samples may attach at random, semi-random or predetermined locations on the support structure. A test sample, such as from a known person or organism, can be exposed to the array or grid, such that complimentary genes or fragments hybridize to probes at the individual sites on a surface of a plate. In certain applications, such as sequencing, templates or fragments of genetic material may be located at the sites, and nucleotides or other molecules may be caused to hybridize to the templates to determine the nature or sequence of the templates. The sites can then be examined by scanning specific frequencies of light over the sites to identify which genes or fragments in the sample were present, by fluorescence of the sites at which genes or fragments hybridized.

These plates are sometimes referred to as microarrays, gene or genome chips, DNA chips, gene arrays, and so forth, and may be used for expression profiling, monitoring expression levels, genotyping, sequencing, and so forth. For example, diagnostic uses may include evaluation of a particular patient's genetic makeup to determine whether a disease state is present or whether pre-disposition for a particular condition exists. The reading and evaluation of such plates are an important aspect of their utility. Although microarrays allow separate biological components to be presented for bulk processing and individual detection, the number of components that can be detected in a single experiment is limited by the resolution of the system. Furthermore, the bulk reagents used in some methods can be expensive such that reduced volumes are desired. While these issues can be addressed by increasing efficiency through imaging multiple surfaces of a single flow cell, multi-surface imaging may be associated with further complications resulting from optical aberrations associated with each of the imaged surfaces. The disclosed techniques provide methods and systems that can allow for high efficiency multi-surface array based detection which have lower cost and complexity than methods and systems which are currently in use. Other advantages are provided as well and will be apparent from the description below.

### SUMMARY

The disclosed technology provides a novel approach to analytical sample imaging and evaluation that reduces the complexity of imaging and evaluation subsystems having multiple surfaces that support samples. The support structure may, for instance, be a flow cell through which a reagent fluid is allowed to flow and interact with biological samples. Excitation radiation from at least one radiation source may be used to excite the biological samples on multiple surfaces. In this manner, fluorescent radiation may be emitted from the biological samples and subsequently captured and detected by detection optics and at least one detector. The returned radiation may then be used to generate image data. This imaging of multiple surfaces may be accomplished either sequentially or simultaneously. In addition, the techniques described herein may be used with any of a variety of types of imaging systems. For instance, both epifluorescent and total internal reflection (TIR) methods may benefit from the disclosed techniques. In addition, the biological samples imaged may be present on the surfaces of the support structure in random locations or in patterns.

An implementation relates to a machine comprising a lens, a flow cell, and a controller. In such an implementation, the lens may have a field of view and be immersed in a first fluid having a first refractive index. Similarly, in such an implementation, the flow cell may comprise first and second surfaces separated by a second fluid having a second refractive index. Additionally, the controller may be to move the lens from a first position having a first distance to the flow cell to a second position having a second distance from the flow cell. The controller may also be to, using the lens, capture light emitted from nucleic acids disposed on the first surface of the flow cell when the lens is separated from the flow cell by the first distance. The controller may also be to, using the lens, capture light emitted from nucleic acids disposed on the second surface of the flow cell when the lens is separated from the flow cell by the second distance. The controller may also be to determine a nucleic acid sequence for a biological sample based on the emitted light. In such an implementation, an optical path difference between marginal light rays and axial light rays in the field of view of the lens when the lens is separated from the flow cell by the first distance may be substantially equal to an optical path difference between marginal light rays and axial light rays in the field of view of the lens when the lens is separated from the flow cell by the second distance.

In some implementations of a machine such as described in the preceding paragraph, the first refractive index and the second refractive index may be substantially equal.

In some implementations of a machine such as described in the preceding paragraph, the lens may be comprised by optics adapted to capture light emitted from nucleic acid sequences disposed on the first surface of the flow cell, and to allow the biological sample to be imaged on a detector with diffraction limited imaging quality. In some such implementations, the first refractive index and the second refractive index are substantially equal means spherical aberration caused by any difference between the first refractive index and the second refractive index is low enough not to prevent diffraction limited imaging of the biological sample based on: light emitted from nucleic acid sequences disposed on the first surface of the flow cell; and light emitted from nucleic acid sequences disposed on the second surface of the flow cell.

In some implementations of a machine such as described in the third paragraph of this summary, the first fluid and the second fluid may be the same fluid.

In some implementations of a machine such as described in the third paragraph of this summary, the first fluid and the second fluid may be different fluids.

In some implementations of a machine such as described in the second paragraph of this summary, the first surface of the flow cell is a top surface of the flow cell, and the second surface of the flow cell is a bottom surface of the flow cell.

In some implementations of a machine such as described in the second paragraph of this summary, the lens may be comprised by optics adapted to capture light emitted from nucleic acid sequences disposed on the first surface of the flow cell, and to allow the biological sample to be imaged on a detector with diffraction limited imaging quality. In some such implementations, the optical path difference between marginal light rays and axial light rays in the field of view of the lens when the lens is separated from the flow cell by the first distance is substantially equal to the difference between marginal light rays and axial light rays in the field of view of the lens when the lens is separated from the flow cell by the second distance means spherical aberration is low enough not to prevent diffraction limited imaging of the biological sample based on: light emitted from nucleic acid sequences disposed on the first surface of the flow cell; and light emitted from nucleic acid sequences disposed on the second surface of the flow cell.

Another implementation relates to a method comprising capturing light emitted from nucleic acids disposed on a first surface of a flow cell using a lens which is a first distance from the flow cell and immersed in a first fluid having a first refractive index. Such a method may also include moving the lens to a position a second distance from the flow cell. Such a method may also include capturing light emitted from nucleic acids disposed on a second surface of the flow cell using the lens immersed in the first fluid having the first refractive index while the lens is at the second distance from the flow cell, wherein the first surface of the flow cell is separated from the second surface of the flow cell by a second fluid having a second refractive index. Such a method may also include determining a nucleic acid sequence for a biological sample based on the light emitted from nucleic acids disposed on the first surface of the flow cell and the light emitted from the nucleic acids disposed on the second surface of the flow cell. In such a method, an optical path difference between marginal light rays and axial light rays in the field of view of the lens when the lens is at the first distance from the flow cell is substantially equal to an optical path difference between marginal light rays and axial light rays in the field of view of the lens when the lens is at the second distance from the flow cell.

In some implementations of a method such as described in the preceding paragraph, the first refractive index and the second refractive index may be substantially equal.

In some implementations of a method such as described in the preceding paragraph, the lens may be comprised by optics adapted to capture light emitted from nucleic acid sequences disposed on the first surface of the flow cell, and to allow the biological sample to be imaged on a detector with diffraction limited imaging quality. In some such implementations, the first refractive index and the second refractive index are substantially equal means spherical aberration caused by any difference between the first refractive index and the second refractive index is low enough not to prevent diffraction limited imaging of the biological sample based on: light emitted from nucleic acid sequences disposed on the first surface of the flow cell; and light emitted from nucleic acid sequences disposed on the second surface of the flow cell.

In some implementations of a method such as described in the tenth paragraph of this summary, the first fluid and the second fluid may be the same fluid.

In some implementations of a method such as described in the tenth paragraph of this summary, the first fluid and the second fluid may be different fluids.

In some implementations of a method such as described in the ninth paragraph of this summary, the first surface of the flow cell is a top surface of the flow cell, and the second surface of the flow cell is a bottom surface of the flow cell.

In some implementations of a method such as described in the ninth paragraph of this summary, the lens is comprised by optics adapted to capture light emitted from nucleic acid sequences disposed on the first surface of the flow cell, and to allow the biological sample to be imaged on a detector with diffraction limited imaging quality. In some such implementations, the optical path difference between marginal light rays and axial light rays in the field of view of the lens when the lens at the first distance from the flow cell is substantially equal to the optical path difference between marginal light rays and axial light rays in the field of view of the lens when the lens at the second distance from the flow cell means spherical aberration is low enough not to prevent diffraction limited imaging of the biological sample based on: light emitted from nucleic acid sequences disposed on the first surface of the flow cell; and light emitted from nucleic acid sequences disposed on the second surface of the flow cell.

Another implementation relates to a non-transitory computer readable medium storing instructions to, when executed by a processor, cause a biological sample imaging system to perform acts. In some such implementations, the acts may comprise capturing light emitted from nucleic acids disposed on a first surface of a flow cell using a lens at a first distance from the flow cell which has a field of view and which is immersed in a first fluid having a first refractive index. In some such implementations, the acts may comprise moving the lens to a position a second distance from the flow cell. In some such implementations, the acts may comprise capturing light emitted from nucleic acids disposed on a second surface of the flow cell using the lens immersed in the first fluid having the first refractive index while the lens is at the second distance from the flow cell, wherein the first surface of the flow cell is separated from the second surface of the flow cell by a second fluid having a second refractive index. In some such implementations, the acts may comprise determining a nucleic acid sequence for a biological sample based on the light emitted from nucleic acids disposed on the first surface of the flow cell and the light emitted from the nucleic acids disposed on the second surface of the flow cell. In some such implementations, an optical path difference between marginal light rays and axial light rays in the field of view of the lens when the lens is at the first distance from the flow cell may be substantially equal to an optical path difference between marginal light rays and axial light rays in the field of view of the lens when the lens is at the second distance from the flow cell.

In some implementations such as described in the preceding paragraph, the first refractive index and the second refractive index may be substantially equal.

In some implementations such as described in the preceding paragraph, the lens may be comprised by optics adapted to capture light emitted from nucleic acid sequences disposed on the first surface of the flow cell, and to allow the biological sample to be imaged on a detector with diffraction limited imaging quality. In some such implementations, the first refractive index and the second refractive index are substantially equal means spherical aberration caused by any difference between the first refractive index and the second refractive index is low enough not to prevent diffraction limited imaging of the biological sample based on: light emitted from nucleic acid sequences disposed on the first surface of the flow cell; and light emitted from nucleic acid sequences disposed on the second surface of the flow cell.

In some implementations such as described in the sixteenth paragraph of this summary, the first surface of the flow cell is a top surface of the flow cell, and the second surface of the flow cell is a bottom surface of the flow cell. In some implementations such as described in the sixteenth paragraph of this summary, the lens is comprised by optics adapted to capture light emitted from nucleic acid sequences disposed on the first surface of the flow cell, and to allow the biological sample to be imaged on a detector with diffraction limited imaging quality. In some such implementations, the optical path difference between marginal light rays and axial light rays in the field of view of the lens when the lens at the first distance from the flow cell is substantially equal to the optical path difference between marginal light rays and axial light rays in the field of view of the lens when the lens at the second distance from the flow cell means spherical aberration is low enough not to prevent diffraction limited imaging of the sample based on: light emitted from nucleic acid sequences disposed on the first surface of the flow cell; and light emitted from nucleic acid sequences disposed on the second surface of the flow cell.

In some implementations such as described in the sixteenth paragraph of this summary, the first fluid and the second fluid may be the same fluid.

Other features and aspects of the disclosed technology will become apparent from the following detailed description, taken in conjunction with the accompanying drawings, which illustrate, by way of example, the features in accordance with examples of the disclosed technology. The summary is not intended to limit the scope of any protection provided by this document or any related document, which scope is defined by the respective document's claims and equivalents.

It should be appreciated that all combinations of the foregoing concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE SEVERAL VIEWS OF THE DRAWINGS

FIG. 1 is a diagrammatical overview for a biological sample imaging system;

FIG. 2 is a diagrammatical perspective view of an exemplary radiation line directed toward a surface of a support structure to semi-confocally irradiate biological sites, and to semi-confocally return radiation to a detector;

FIG. 3 is a sectional view of an exemplary support structure with excitation radiation directed at two surfaces of the support structure;

FIG. 4 is a diagrammatical perspective view of an exemplary support structure having an array of biological component sites in a spatially ordered pattern;

FIG. 5 is a diagrammatical perspective view of an exemplary support structure having biological component sites in a random spatial distribution;

FIG. 6 is a sectional view of an exemplary support structure with excitation radiation directed at multiple surfaces of the support structure;

FIG. 7 illustrates exemplary dimensions between an objective and support structure which may exist in some implementations;

FIG. 8 illustrates exemplary ray tracing from a light source to the first lens of an objective for multiple surface imaging; and

FIG. 9 illustrates exemplary images expected for top and bottom surface imaging when the imaging system is optimized for top surface imaging.

### DETAILED DESCRIPTION

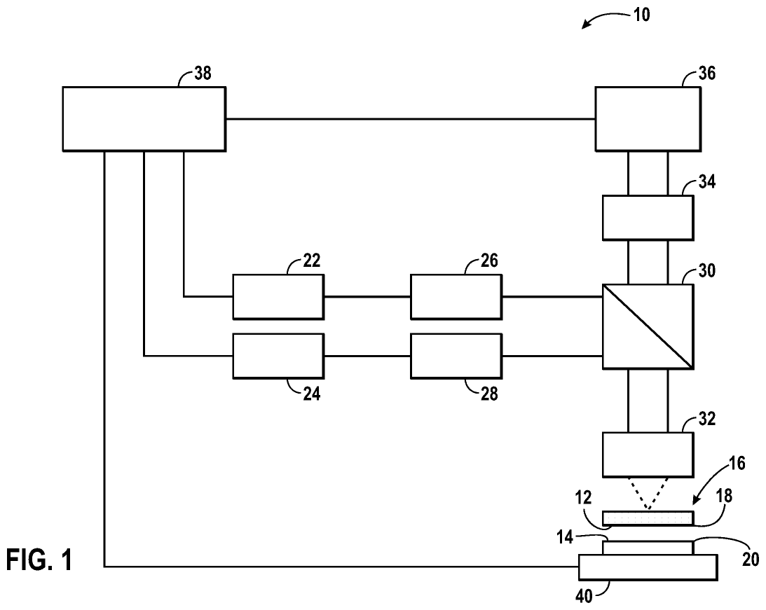

Turning now to the drawings, and referring first to FIG. 1, a biological sample imaging system 10 is illustrated diagrammatically. The biological sample imaging system 10 is capable of imaging multiple biological components 12, 14 within a support structure 16. For instance, in the illustrated embodiment, a first biological component 12 may be present on a first surface 18 of the support structure 16 while a second biological component 14 may be present on a second surface 20 of the support structure. The support structure 16 may, for instance, be a flow cell with an array of biological components 12, 14 on the interior surfaces 18, 20 which generally mutually face each other and through which reagents, flushes, and other fluids may be introduced, such as for binding nucleotides or other molecules to the sites of biological components 12, 14. Fluorescent tags on the molecules that bind to the components may, for instance, include dyes that fluoresce when excited by appropriate excitation radiation. Assay methods that include the use of fluorescent tags and that can be used in an apparatus or method set forth herein include those set forth elsewhere herein such as genotyping assays, gene expression analysis, methylation analysis, or nucleic acid sequencing analysis.

Those skilled in the art will recognize that a flow cell may be used with any of a variety of arrays known in the art to achieve similar results. Such arrays may be formed by disposing the biological components of samples randomly or in predefined patterns on the surfaces of the support using various techniques. In a particular embodiment, clustered arrays of nucleic acid colonies can be prepared as described in U.S. Pat. No. 7,115,400; U.S. Patent Application Publication No. 2005/0100900; PCT Publication No. WO 00/18957; or PCT Publication No. WO 98/44151, each of which is hereby incorporated by reference. Such methods are known as bridge amplification or solid-phase amplification and are particularly useful for sequencing applications.

Other exemplary random arrays that can be used include, without limitation, those in which beads are associated with a solid support, examples of which are described in U.S. Pat. Nos. 6,355,431; 6,327,410; and 6,770,441; U.S. Patent Application Publication Nos. 2004/0185483 and US 2002/0102578; and PCT Publication No. WO 00/63437, each of which is hereby incorporated by reference. Beads can be located at discrete locations, such as wells, on a solid-phase support, whereby each location accommodates a single bead.

Any of a variety of other arrays known in the art or methods for fabricating such arrays can be used. Commercially available microarrays that can be used include, for example, an Affymetrix® GeneChip® microarray or other microarray synthesized in accordance with techniques sometimes referred to as VLSIPS™ (Very Large Scale Immobilized Polymer Synthesis) technologies as described, for example, in U.S. Pat. Nos. 5,324,633; 5,744,305; 5,451,683; 5,482,867; 5,491,074; 5,624,711; 5,795,716; 5,831,070; 5,856,101; 5,858,659; 5,874,219; 5,968,740; 5,974,164; 5,981,185; 5,981,956; 6,025,601; 6,033,860; 6,090,555; 6,136,269; 6,022,963; 6,083,697; 6,291,183; 6,309,831; 6,416,949; 6,428,752; and 6,482,591, each of which is hereby incorporated by reference. A spotted microarray can also be used. An exemplary spotted microarray is a CodeLink™ Array available from Amersham Biosciences. Another microarray that is useful is one that is manufactured using inkjet printing methods such as SurePrint™ Technology available from Agilent Technologies.

Sites or features of an array are typically discrete, being separated with spaces between each other. The size of the sites and/or spacing between the sites can vary. For example, an array useful in conjunction with the disclosed techniques can have sites that are separated by less than 100 μm, 50 μm, 10 μm, 5 μm, 1 μm or 0.5 μm (e.g., 350 nm, or 10 nm). An apparatus or method implemented based on this disclosure can be used to image an array at a resolution sufficient to distinguish sites at the above densities or density ranges.

As exemplified herein, a surface used in an apparatus or method implemented based on this disclosure is typically a manufactured surface. It is also possible to use a natural surface or a surface of a natural support structure; however the disclosed technology may be applied in embodiments where the surface is not a natural material nor a surface of a natural support structure. Accordingly, components of biological samples can be removed from their native environment and attached to a manufactured surface.

Any of a variety of biological components can be present on a surface. Exemplary components include, without limitation, nucleic acids such as DNA or RNA, proteins such as enzymes or receptors, polypeptides, nucleotides, amino acids, saccharides, cofactors, metabolites or derivatives of these natural components. Although the apparatus and methods described herein are exemplified with respect to components of biological samples, it will be understood that other samples or components can be used as well. For example, synthetic samples can be used such as combinatorial libraries, or libraries of compounds having species known or suspected of having a desired structure or function. Thus, the apparatus or methods can be used to synthesize a collection of compounds and/or screen a collection of compounds for a desired structure or function.

Returning to the exemplary system of FIG. 1, the biological sample imaging system 10 may include at least a first radiation source 22 but may also include a second radiation source 24 (or additional sources). The radiation sources 22, 24 may be lasers operating at different wavelengths. The selection of the wavelengths for the lasers will typically depend upon the fluorescence properties of the dyes used to image the component sites. Multiple different wavelengths of the lasers used may permit differentiation of the dyes at the various sites within the support structure 16, and imaging may proceed by successive acquisition of a series of images to enable identification of the molecules at the component sites in accordance with image processing and reading logic generally known in the art. In other implementations, light emitting diodes (LEDs) may be utilized as radiation sources 22, 24. Other radiation sources known in the art can be used including, for example, an arc lamp or quartz halogen lamp. Particularly useful radiation sources are those that produce electromagnetic radiation in the ultraviolet (UV) range (about 200 to 390 nm), visible (VIS) range (about 390 to 770 nm), infrared (IR) range (about 0.77 to 25 microns), or other range of the electromagnetic spectrum.

For ease of description, embodiments utilizing fluorescence-based detection are used as examples. However, it will be understood that other detection methods can be used in connection with the apparatus and methods set forth herein. For example, a variety of different emission types can be detected such as fluorescence, luminescence, or chemiluminescence. Accordingly, components to be detected can be labeled with compounds or moieties that are fluorescent, luminescent, or chemiluminescent. Signals other than optical signals can also be detected from multiple surfaces using apparatus and methods that are analogous to those exemplified herein with the exception of being modified to accommodate the particular physical properties of the signal to be detected.

Output from the radiation sources 22, 24 may be directed through conditioning optics 26, 28 for filtering and shaping of the beams. For example, in a presently contemplated embodiment, the conditioning optics 26, 28 may generate a generally linear beam of radiation, and combine beams from multiple lasers, for example, as described in U.S. Pat. No. 7,329,860. The laser modules can additionally include a measuring component that records the power of each laser. The measurement of power may be used as a feedback mechanism to control the length of time an image is recorded in order to obtain uniform exposure, and therefore more readily comparable signals. In other implementations, the conditioning optics 26, 28 may form a rectilinear beam of radiation, such as a square or rectangular shape.

After passing through the conditioning optics 26, 28, the beams may be directed toward directing optics 30 which redirect the beams from the radiation sources 22, 24 toward focusing optics 32. The directing optics 30 may include a dichroic mirror configured to redirect the beams toward the focusing optics 32 while also allowing certain wavelengths of a retrobeam to pass therethrough. The focusing optics 32 may confocally direct radiation to one or more surfaces 18, 20 of the support structure 16 upon which individual biological components 12, 14 are located. For instance, the focusing optics 32 may include a microscope objective that confocally directs and concentrates the radiation sources 22, 24 along a line to a surface 18, 20 of the support structure 16. In some implementations, the focusing optics 32 may focus the radiation sources 22, 24 above or below the surface 18, 20 such that the concentration is below a photosaturation threshold of the component to be excited and/or a threshold to reduce damage to a sample. In such an implementation, the focusing optics 32 can also maintain the focal plane of a detector 36 at the plane of the surface 18, 20 while the excitation radiation is defocused relative to the plane of the surface 18, 20. In some implementations, the focusing optics 32 can be moveable relative to the surfaces 18, 20. For example, the focusing optics can be mounted to a z-stage, such as a voice coil or piezoelectric component, to move the focusing optics 32 through the axis perpendicular to the plane of the surface 18, 20. In some implementations, the focusing optics 32 can be moveable along the x and/or y axis relative to the surface 18, 20.

Biological component sites on the support structure 16 may use one or more reagents that fluoresce at particular wavelengths in response to an excitation beam and thereby return radiation for imaging. For instance, the fluorescent components may be generated by fluorescently tagged nucleotides that hybridize to a complementary nucleotide of the sample using a polymerase. In other implementations, the fluorescently tagged nucleotides may temporarily associate with the complementary nucleotide of the sample and are thereafter removed. In still further implementations, a fluorescently tagged oligonucleotide can be used. As noted above, the fluorescent properties of these components may be changed through the introduction of reagents into the support structure 16 (e.g., by cleaving the dye from the molecule, blocking attachment of additional molecules, adding a quenching reagent, adding an acceptor of energy transfer, and so forth). As will be appreciated by those skilled in the art, the wavelength at which the dyes of the sample are excited and the wavelength at which they fluoresce will depend upon the absorption and emission spectra of the specific dyes. Such returned radiation may propagate back through the directing optics 30. This retrobeam may generally be directed toward detection optics 34 which may filter the beam such as to separate different wavelengths within the retrobeam, and direct the retrobeam toward at least one detector 36.

The detector 36 may be based upon any suitable technology, and may be, for example, a charged coupled device (CCD) sensor that generates pixilated image data based upon photons impacting locations in the device. However, it will be understood that any of a variety of other detectors may also be used including, but not limited to, a detector array configured for time delay integration (TDI) operation, a complementary metal oxide semiconductor (CMOS) detector, an avalanche photodiode (APD) detector, a Geiger-mode photon counter, or any other suitable detector. TDI mode detection can be coupled with line scanning as described in U.S. Pat. No. 7,329,860.

The detector 36 may generate image data, for example, at a resolution between 0.1 and 50 microns, which is then forwarded to a control/processing system 38. In general, the control/processing system 38 may perform various operations, such as analog-to-digital conversion, scaling, filtering, and association of the data in multiple frames to appropriately and accurately image multiple sites at specific locations on a sample. The control/processing system 38 may store the image data and may ultimately forward the image data to a post-processing system (not shown) where the data are analyzed. Depending upon the types of sample, the reagents used, and the processing performed, a number of different uses may be made of the image data. For example, nucleotide sequence data can be derived from the image data, or the data may be employed to determine the presence of a particular gene, characterize one or more molecules at the component sites, and so forth. The operation of the various components illustrated in FIG. 1 may also be coordinated with the control/processing system 38. In a practical application, the control/processing system 38 may include hardware, firmware, and software designed to control operation of the radiation sources 22, 24, movement and focusing of the focusing optics 32, a translation system 40, and the detection optics 34, and acquisition and processing of signals from the detector 36. The control/processing system 38 may thus store processed data and further process the data for generating a reconstructed image of irradiated sites that fluoresce within the support structure 16. The image data may be analyzed by the system itself, or may be stored for analysis by other systems and at different times subsequent to imaging.

The support structure 16 may be supported on a translation system 40 which allows for focusing and movement of the support structure 16 before and during imaging. The stage may be configured to move the support structure 16, thereby changing the relative positions of the radiation sources 22, 24 and detector 36 with respect to the surface bound biological components for progressive scanning. Movement of the translation system 40 can be in one or more dimensions including, for example, one or both of the dimensions that are orthogonal to the direction of propagation for the excitation radiation line, typically denoted as the X and Y dimensions. In particular embodiments, the translation system 40 may be configured to move in a direction perpendicular to the scan axis for a detector array. A translation system 40 may be further configured for movement in the dimension along which the excitation radiation line propagates, typically denoted as the Z dimension. Movement in the Z dimension can also be useful for focusing.

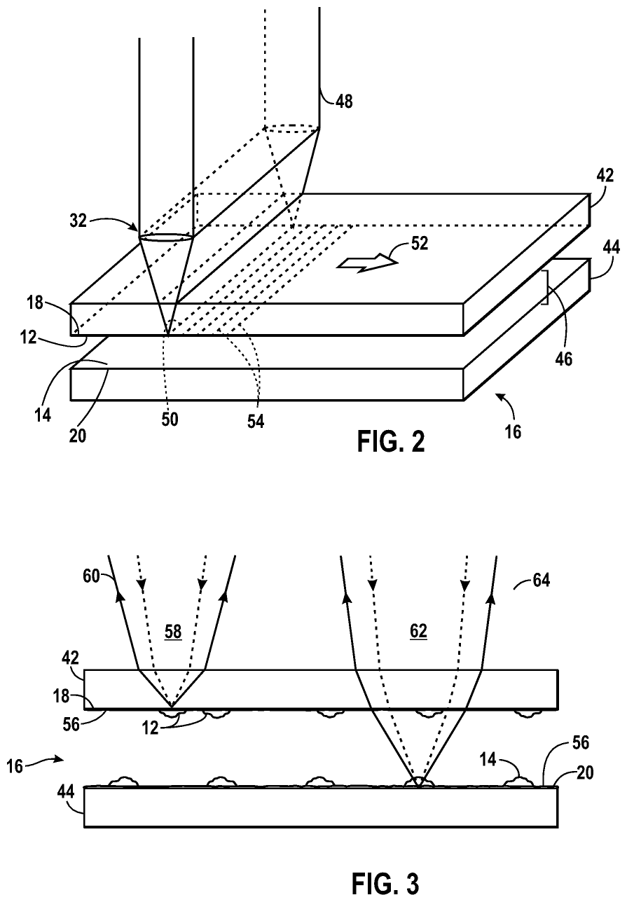

FIG. 2 is a diagrammatical representation of an exemplary semi-confocal line scanning approach to imaging the support structure 16. In the illustrated embodiment, the support structure 16 includes an upper plate 42 and a lower plate 44 with an internal volume 46 between the upper and lower plates 42, 44. The upper and lower plates 42, 44 may be made of any of a variety of materials but may preferably be made of a substrate material that is substantially transparent at the wavelengths of the excitation radiation and the fluoresced retrobeam, allowing for the passage of excitation radiation and returned fluorescent emissions without significant loss of signal quality. Moreover, when used in epifluorescent imaging arrangements as shown, one of the surfaces through which the radiation traverses may be substantially transparent at the relevant wavelengths, while the other (which is not traversed by radiation) may be less transparent, translucent, or even opaque or reflective. The upper and lower plates 42, 44 may include polymer materials or other intermediary components and may further both contain biological components 12, 14 on their respective, inwardly facing surfaces 18, 20. As discussed above, the internal volume 46 may, for instance, include one or more internal passages of a flow cell though which reagent fluids may flow.

The reactive elements, such as fluorinated nucleotides, associated with the support structure 16 may be irradiated by excitation radiation 48 along a radiation line 50. The radiation line 50 may be formed by the excitation radiation 48 from the radiation sources 22, 24, directed by the directing optics 30 through the focusing optics 32. The radiation sources 22, 24 may generate beams that are processed and shaped to provide a linear cross section or radiation line including a plurality of wavelengths of radiation used to cause fluorescence at correspondingly different wavelengths from the biological components 12, 14, depending upon the particular dyes used. The focusing optics 32 may then semi-confocally direct the excitation radiation 48 toward the first surface 18 of the support structure 16 to irradiate sites of fluorinated nucleotides associated with the biological component 12 along the radiation line 50. In addition, the support structure 16, the directing optics 30, the focusing optics 32, or some combination thereof, may be slowly translated such that the resulting radiation line 50 progressively irradiates the component as indicated by the arrow 52. This translation results in successive scanning of regions 54 which allow for the gradual irradiation of the fluorinated nucleotides associated with the biological components 12 of the entire first surface 18 of the support structure 16. As will be discussed in more detail below, the same process may also be used to gradually irradiate the second surface 20 of the support structure 16. Indeed, the process may be used for multiple surfaces within the support structure 16.

Exemplary methods and apparatus for line scanning are described in U.S. Pat. No. 7,329,860, which is incorporated herein by reference, and which describes a line scanning apparatus having a detector array configured to achieve confocality in the scanning axis by restricting the scan-axis dimension of the detector array. More specifically, the scanning device can be configured such that the detector array has rectangular dimensions such that the shorter dimension of the detector is in the scan-axis dimension and imaging optics are placed to direct a rectangular image of a sample region to the detector array such that the shorter dimension of the image is also in the scan-axis dimension. In this way, semi-confocality can be achieved since confocality occurs in a single axis (i.e., the scan axis). Thus, detection is specific for features on the surface of a substrate, thereby rejecting signals that may arise from the solution around the feature. The apparatus and methods described in U.S. Pat. No. 7,329,860 can be modified such that two or more surfaces of a support are scanned in accordance with the description herein.

Detection apparatus and methods other than line scanning can also be used. For example, point scanning can be used as described below or in U.S. Pat. No. 5,646,411, which is incorporated herein by reference. Wide angle area detection can be used with or without scanning motion. As set forth in further detail elsewhere herein, TIR methods can also be used.

As illustrated generally in FIG. 2, the radiation line 50 used to image the sites of biological components 12, 14 may be a continuous or discontinuous line. As such, some embodiments may include a discontinuous line made up of a plurality of confocally or semi-confocally directed beams of radiation which nevertheless irradiate a plurality of points along the radiation line 50. These discontinuous beams may be created by one or more sources that are positioned or scanned to provide the excitation radiation 48. These beams, as before, may be confocally or semi-confocally directed toward the first or second surfaces 18, 20 of the support structure 16 to irradiate sites of biological component 12, 14. As with the continuous semi-confocal line scanning described above, the support structure 16, the directing optics 30, the focusing optics 32, or some combination thereof, may be advanced slowly as indicated by arrow 52 to irradiate successive scanned regions 54 along the first or second surfaces 18, 20 of the support structure 16, and thereby successive regions of the sites of biological components 12, 14.

It should be noted that the system will typically form and direct excitation and returned radiation simultaneously for imaging. In some embodiments, confocal point scanning may be used such that the optical system directs an excitation point or spot across a biological component by scanning the excitation beam through an objective lens. The detection system images the emission from the excited point on the detector without “descanning” the retrobeam. This occurs since the retrobeam is collected by the objective lens and is split off the excitation beam optical path before returning back through the scan means. Therefore, the retrobeam will appear on the detector 36 at different points depending on the field angle of the original excitation spot in the objective lens. The image of the excitation point, at the detector 36, will appear in the shape of a line as the excitation point is scanned across the sample. This architecture is useful, for example, if the scan means cannot for some reason accept the retrobeam from the sample. Examples are holographic and acoustic optic scan means that are able to scan a beam at very high speeds but utilize diffraction to create the scan. Therefore, the scan properties are a function of wavelength. The retrobeam of emitted radiation is at a different wavelength from the excitation beam. Alternatively or additionally, emission signals may be collected sequentially following sequential excitation at different wavelengths.

In particular embodiments, an apparatus or method can detect features on a surface at a rate of at least about 0.01 mm²/sec. Depending upon the particular application, faster rates can also be used including, for example, in terms of the area scanned or otherwise detected, a rate of at least about 0.02 mm²/sec, 0.05 mm²/sec, 0.1 mm²/sec, 1 mm²/sec, 1.5 mm²/sec, 5 mm²/sec, 10 mm²/sec, 50 mm²/sec, 100 mm²/sec, or faster. If desired, for example, to reduce noise, the detection rate can have an upper limit of about 0.05 mm²/sec, 0.1 mm²/sec, 1 mm²/sec, 1.5 mm²/sec, 5 mm²/sec, 10 mm²/sec, 50 mm²/sec, or 100 mm²/sec.

In some instances, the support structure 16 may be used in such a way that biological components are expected to be present on only one surface. However, in some instances, biological material is present on multiple surfaces within the support structure 16. For instance, FIG. 3 illustrates a typical support structure 16 where biological material has attached to the first surface 18 as well as to the second surface 20. In the illustrated embodiment, an attachment layer 56 has formed on both the first surface 18 and the second surface 20 of the support structure 16. A first excitation radiation 58 source may be used to irradiate one of many sites of biological component 12 on the first surface 18 of the support structure 16 and return a first fluorescent emission 60 from a fluorinated nucleotide associated with the irradiated biological component 12. Simultaneously or sequentially, a second source of excitation radiation 62 may be used to irradiate one of many sites of biological component 14 on the second surface 20 of the support structure 16, and return a second fluorescent emission 64 from the irradiated biological component 14.

Although the embodiment exemplified in FIG. 3 shows excitation from source 58 and source 62 coming from the same side of the support structure 16, it will be understood that the optical system can be configured to impinge on the surfaces from opposite sides of the support structure 16. Taking FIG. 3 as an example, upper surface 18 can be irradiated from excitation source 58 as shown and the lower surface 20 can be irradiated from below. Similarly, emission can be detected from one or more sides of a support structure. In particular embodiments, different sides of the support structure 16 can be excited from the same radiation source by first irradiating one side and then flipping the support structure to bring another side into position for excitation by the radiation source.

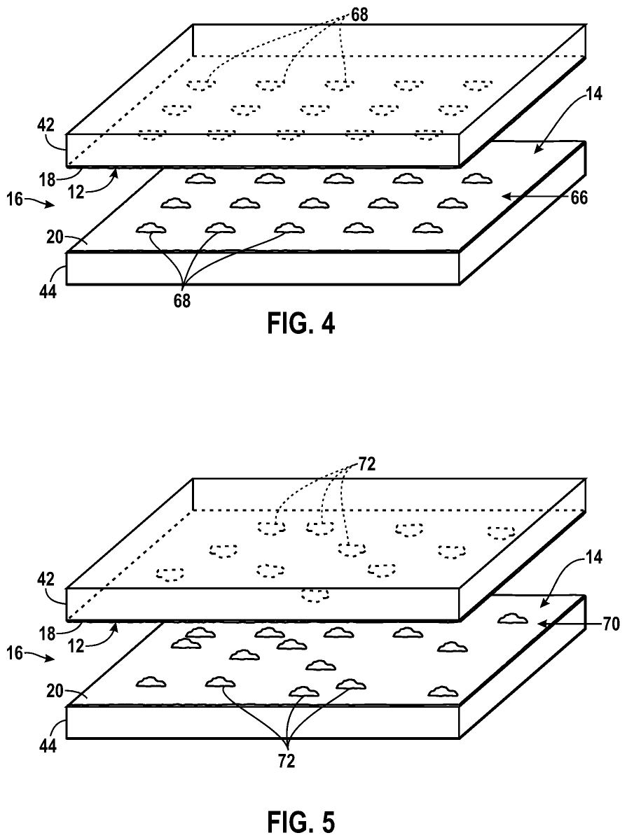

The distribution of biological components 12, 14 may follow many different patterns. For instance, FIG. 4 illustrates a support structure 16 where the biological components 12, 14 at sites or features on the first and second surfaces 18, 20 are distributed evenly in a spatially ordered pattern 66 of biological component sites 68. For example, certain types of microarrays may be used where the location of individual biological component sites 68 may be in a regular spatial pattern, such as a hexagonal pattern. The pattern can include sites at pre-defined locations. In contrast, in other types of biological imaging arrays, biological components attach to surfaces at sites that occur in random or statistically varying positions such that imaging the microarray is used to determine the location of each of the individual biological component features. Thus, the pattern of features need not be pre-determined despite being the product of a synthetic or manufacturing process.

For instance, FIG. 5 illustrates a support structure 16 where the sites on the first and second surfaces 18, 20 are located in a random spatial distribution 70 of biological component sites 72. However, with both fixed arrays 66 and random distribution 70 of biological sample sites, imaging of multiple surfaces 18, 20 of the support structure 16 may be possible. In addition, it should be noted that in both instances, the biological components at the individual sites may constitute either a population of identical molecules or a random mix of different molecules. Furthermore, the density of biological samples may vary and may be at least 1,000 sites per square millimeter.

The present techniques accommodate such varied physical arrangements of the multiple surfaces within the support structure 16, as well as the varied disposition of the sites within components on the surfaces. As discussed above with reference to FIGS. 2 and 3, in the embodiments with a support structure 16 having a first surface 18 and a second surface 20, a first source of excitation radiation 58 may irradiate sites of biological component 12 on the first surface 18, and return a first fluorescent emission 60, while a second source of excitation radiation 62 may irradiate sites of biological component 14 on the second surface 20 and return a second fluorescent emission 64 source, as illustrated in FIG. 3. Thus, components of the volume of sample between two surfaces need not be detected and can be rejected. Selective detection of a surface of a support structure provides preferential detection of the surface compared to the volume of the support structure adjacent the surface and compared to one or more other surfaces of the support structure.

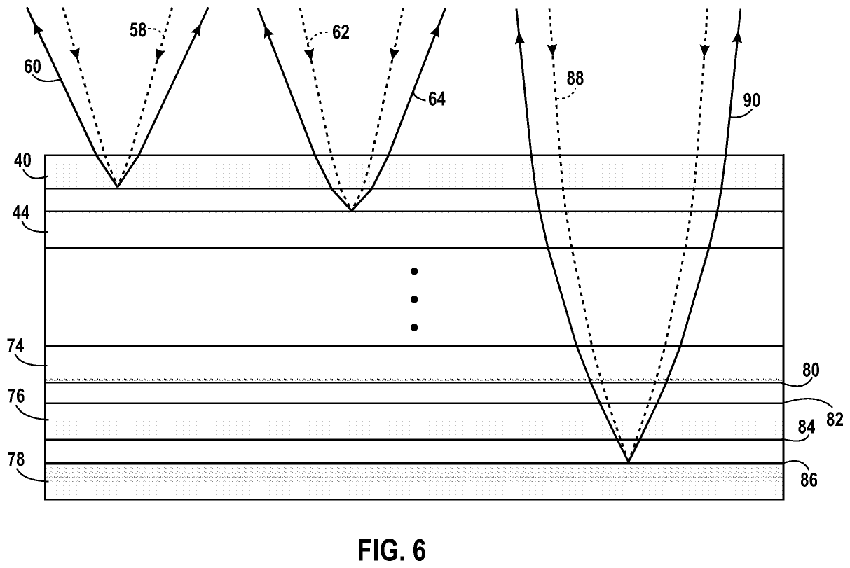

In more complex configurations, it may be useful to irradiate more than two surfaces. For instance, FIG. 6 illustrates a support structure 16 having N number of plates including a first plate 42, a second plate 44, . . . , an N−2 plate 74, an N−1 plate 76, and an N plate 78. These plates define M number of surfaces including a first surface 18, a second surface 20, . . . , an M−3 surface 80, an M−2 surface 82, an M−1 surface 84, and an M surface 86. In the illustrated embodiment, not only the first surface 18 and the second surface 20 of the support structure 16 may be irradiated but, rather, all M number of surfaces may be irradiated. For instance, a source of excitation radiation 88 may be used to irradiate biological component sites on the M^{th} surface 86 of the support structure 16 and return a fluorescent emission 90 from the irradiated biological component. For support structures having a plurality of surfaces it may be desirable to excite upper layers from the top and lower layers from the bottom to reduce photobleaching. Thus, components on layers that are closer to a first exterior side of a support structure can be irradiated from the first side, whereas irradiation from the opposite exterior side can be used to excite components present on layers that are closer to the opposite exterior side.

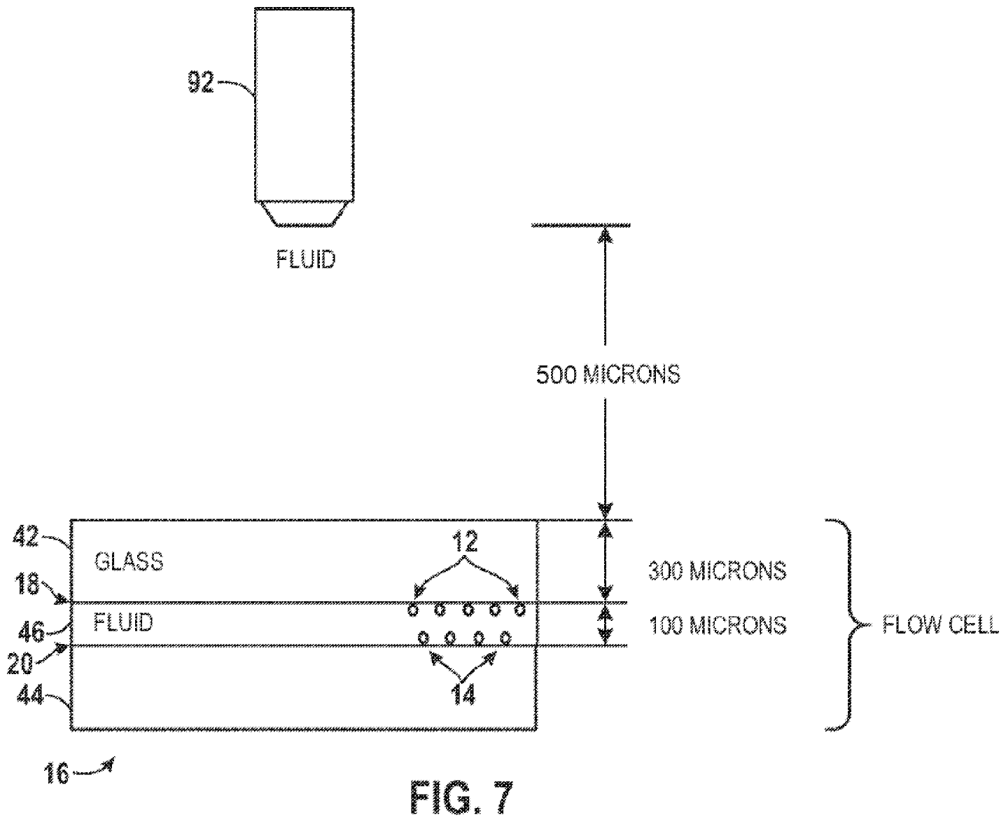

FIG. 7 illustrates an objective 92 through which radiation from emissive biological components 12, 14 on first and second surfaces 18, 20, respectively, of the support structure 16 may be detected. The objective 92 may be one of the components of the focusing optics 32 described above. Although not drawn to scale, FIG. 7 illustrates exemplary dimensions between the objective 92 and the support structure 16. For instance, the objective 92 may be spaced approximately 500 microns from the upper plate 42 of the support structure 16. The biological sample imaging system 10 may be configured to detect emitted radiation from biological components 12 on the first surface 18 through the upper plate 42 which may, for instance, be made of glass having a thickness of from 300 μm to 700 μm. In addition, the biological sample imaging system 10 may also be configured to detect emitted radiation from biological components 14 on the second surface 20 through the upper plate 42 (which may be, for example, from 300-700 μm) plus the fluid (e.g., a reagent solution, such as a buffer solution, an imaging solution, or other reagent) within the internal volume 46 of the support structure 46, whose thickness may vary from 75-100 μm, as shown.

In certain embodiments, the objective 92 may be a plano-convex (PCX) lens whose flat surface faces the sample to avoid turbulence of liquid flow when the objective is used for liquid immersion imaging. In such embodiments, the objective 92 may be immersed in fluid, which may allow it to have a significantly higher numerical aperture than would be possible for a dry objective, since the numerical aperture will be linearly proportional to the refractive index of the media surrounding the objective. This, in turn, may increase the throughput of a system implemented based on this disclosure, since the throughput will be proportional to the square of the numerical aperture.

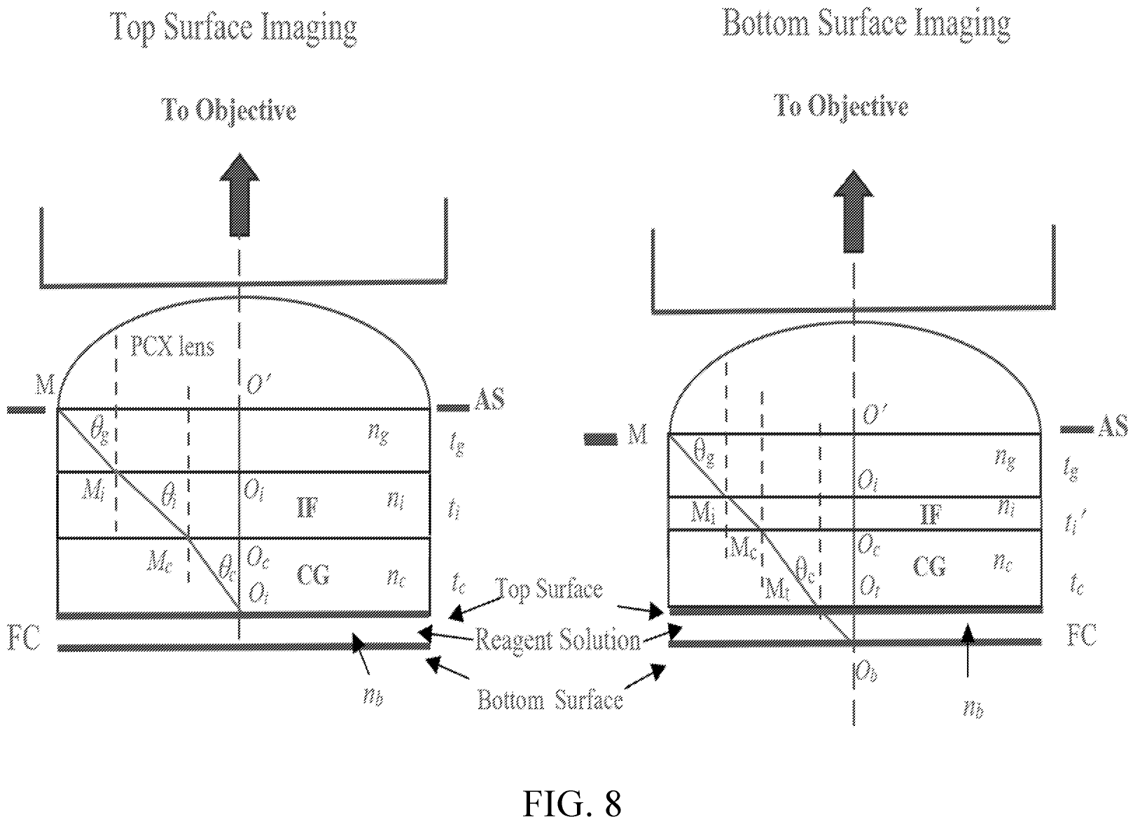

While liquid immersion can allow for increases in numerical aperture, it can also increase aberrations, such as wavefront errors (e.g., spherical aberrations) when performing multi-surface imaging. This can be seen in FIG. 8, which shows ray tracing from a light source to the first lens of the objective for top surface 801 and bottom surface 802 imaging in a dual surface imaging configuration. As shown in that figure, the length of segment M_{c}Mᵢ on the marginal ray will increase with the angle θᵢ in both top and bottom surface imaging. Accordingly, since the maximum wavefront error can be evaluated by the optical path difference between axial and marginal rays, the greater the refractive index of the medium separating the objective from the top surface (nᵢ), the greater the angle θᵢ, and therefore the greater the error in sequencing using the depicted optical layout.

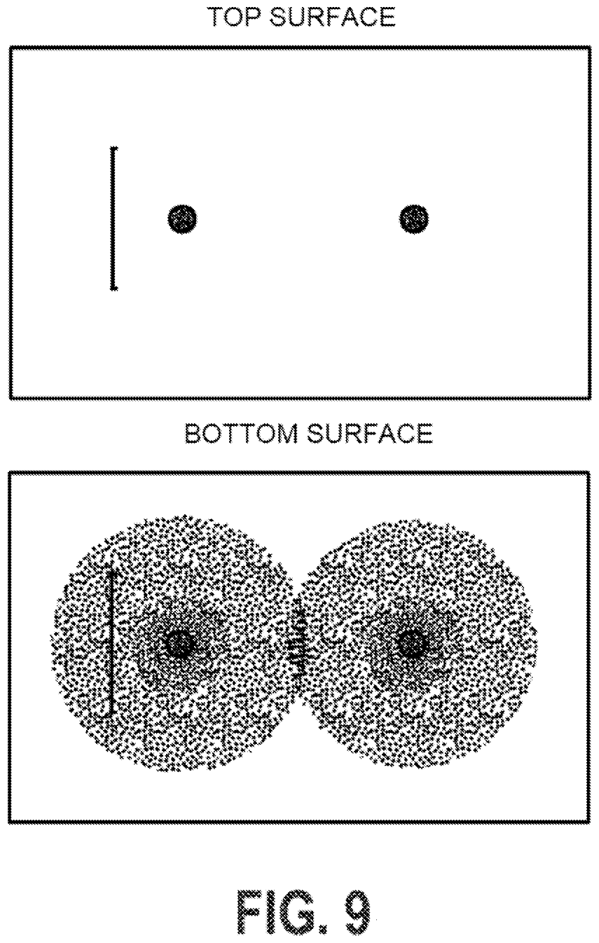

While liquid immersion increases wavefront error, this error is predictable, and so can be compensated for through the design of the objective 92. For example, by designing the objective to compensate for the wavefront error that would be expected for top surface imaging, it is possible to obtain diffraction limited quality images for the top surface. However, because, as shown in FIG. 8, optical paths taken by light when imaging on different surfaces of the support structure 16 are different, optimizing for imaging on a first surface 18 would result in degradation of images from a second surface 20. This is shown graphically in FIG. 9, which illustrates the exemplary images expected for the first and second surfaces 18, 20 of the support structure 16 corresponding to the thickness of the upper plate 42 (e.g., 300 microns) in the configuration shown in FIG. 7, where the imaging system is optimized for the first surface 18. As shown, the imaging system is capable of providing high image quality on the first surface 18 since, in the example of FIG. 9, it was designed to do so. However, in a system optimized for imaging from a first surface, the images taken for the second surface 20 may contain aberrations. Similarly, if the system is optimized for imaging from the second surface 20, images taken from the first surface 18 may contain aberrations.

Errors which may be encountered in imaging from one surface by a system which is optimized for imaging from a different surface are expressed mathematically in equations 1-3, below. In those equations, Wₜₒₚ is the wavefront error for the top surface, W_{bottom} is the wavefront error for the bottom surface, n_{c} is the refractive index of the cover glass, nᵢ is the refractive index of the immersion fluid, n_{g} is the refractive index of the first lens of the objective, n_{b} is the refractive index of the reagent solution, θ_{c} is an angle between the marginal and axial rays through the cover glass, θᵢ is the angle between the marginal and axial rays through the immersion fluid, θ_{g} is the angle between the marginal and axial rays through the first lens of the objective, θ_{b} is the angle between marginal and axial rays through the reagent solution, t_{c} is the thickness of the cover glass, tᵢ is the thickness of the immersion fluid for top surface imaging, tᵢ′ is the thickness of immersion fluid for bottom surface imaging, t_{g} is the thickness of the first lens of the objective, t_{b} is the thickness of the reagent solution, and Δ is the wavefront error difference between the top and bottom surfaces (e.g., the aberration associated with bottom surface imaging in a system optimized for top surface imaging).

$$\begin{matrix}{W}_{top} = {n}_{c} {t}_{c} ( \frac{1}{cos {θ}_{c}} - 1 ) + {n}_{i} {t}_{i} ( \frac{1}{cos {θ}_{i}} - 1 ) + {n}_{g} {t}_{g} ( \frac{1}{cos {θ}_{g}} - 1 ) & Equation 1 \\ {W}_{bottom} = {n}_{b} {t}_{b} ( \frac{1}{cos {θ}_{b}} - 1 ) + {n}_{c} {t}_{c} ( \frac{1}{cos {θ}_{c}} - 1 ) + {n}_{i} {t}_{i} ( \frac{1}{cos {θ}_{i}} - 1 ) + {n}_{g} {t}_{g} ( \frac{1}{cos {θ}_{g}} - 1 ) & Equation 2 \\ Δ = {W}_{bottom} - {W}_{top} = {n}_{b} {t}_{b} ( \frac{1}{cos {θ}_{b}} - 1 ) + {n}_{i} ( {t}_{i}^{′} - {t}_{i} ) ( \frac{1}{cos {θ}_{i}} - 1 ) & Equation 3\end{matrix}$$

A variety of approaches can be used to minimize the impact of this spherical aberration: For example, it is possible to use a compensator, such as described in U.S. Pat. No. RE48,561, which is hereby incorporated by reference in its entirety. However, for some systems aberrations can be significant enough that the expense and/or complexity of a compensator based system may become an obstacle. Accordingly, in some embodiments, either in addition to, or as an alternative to, incorporating a compensator into an imaging system, aberrations may be addressed through coordinating selection of the immersion fluid with the movement of the objective 92 between imaging of different surfaces (e.g., top and bottom surfaces) such that the value of Δ in equation 3 would drop to 0 given the values of n_{b}, t_{b} and θ_{b} associated with the system. To illustrate, consider a case where the immersion fluid is selected to have the same index of refraction as the reagent solution in the internal volume 46 of the support structure 16 (e.g., n_{b}=1.36). This may be done, for example, in some systems which use the same fluid as both the buffer fluid and the immersion fluid and which hold the temperature of those fluids constant, which use water with saline or other organic compound dissolved therein to obtain the appropriate refractive index, or in other types of systems (e.g., systems which use a matching liquid). In this case, n_{b} would be equal to nᵢ, and θ_{b} would be equal to θᵢ, thereby allowing equation 5 to be simplified to equation 4, below.

$$Δ = {n}_{b} ( \frac{1}{cos {θ}_{b}} - 1 ) ( {t}_{b} + {t}_{i}^{′} - {t}_{i} ) \qquad Equation 4$$

Using that equation, it is possible to achieve a value of 0 for Δ (i.e., to obtain the same image quality for the bottom surface as the top surface) by setting tᵢ′ equal to tᵢ−t_{b}. Accordingly, an imaging system can obtain diffraction limited image quality for both top and bottom surfaces of a flow cell by using an immersion fluid with a refractive index equal to its reagent solution, performing a first imaging run of the top surface with the objective separated from the cover glass by a distance of tᵢ, then performing a second imaging run of the bottom surface with the objective separated from the cover glass by a distance equal to tᵢ minus t_{b}.

Other approaches are also possible beyond the compensator based approaches described in RE48,561 or matching n_{b} and nᵢ and setting tᵢ′ so that Δ is reduced to 0. For example, in some cases rather than having the difference between n_{b} and nᵢ be equal to 0, a system implemented based on this disclosure use an immersion liquid with a value of nᵢ which is within some tolerance of n_{b} such that sufficient image quality may still be obtained given the application for which the system is employed. To illustrate how this type of approach may be implemented in practice, consider a system having a numerical aperture of 1.2 which is able to achieve diffraction limited performance so long as the combination of primary and high-order spherical aberrations is less than or equal to 0.07 waves. In this case, the high and low order spherical aberrations created by a mismatch between refractive indices of the buffer and immersion fluids can be calculated by applying Zernike analysis using equations 5 and 6, below.

$$\begin{matrix}W ( ρ , ψ ) = {\sum }_{q - 0}^{∞} {\sum }_{m = 0}^{q} {{c}_{qm} [ \frac{2 ( q + 1 )}{1 + {δ}_{m 0}} ]}^{\frac{1}{2}} {R}_{q}^{m} ( ρ ) cos ( m ψ ) & Equation 5 \\ {R}_{q}^{m} ( ρ ) = {\sum }_{s = 0}^{( q - m ) / 2} \frac{{( - 1 )}^{s} * ( q - s ) !}{S ! * ( \frac{q + m}{2} - s ) ! * ( \frac{q - m}{2} - s ) !} {ρ}^{q - 2 s} & Equation 6\end{matrix}$$

In equations 5 and 6, ρ is the value of sin(ψ) in the unit circle, ψ is the angle equal to θ_{b}−θᵢ, c_{qm} is the Zernike expansion coefficient corresponding to the Zernike radial variable R_{q}^{m}(φ, and δₘ₀ is a normalization variable equal to 1 if m is 0, and 0 otherwise. Applying this analysis to the exemplary 1.2NA system provides the primary and high order spherical aberration information listed below in table 1 for various illustrative theoretical immersion fluids having the specified differences in refractive index (i.e., Δn) from the buffer fluid used in the system.

TABLE 1

| Immersion | | | | | | | | | | | | |
|---|---|---|---|---|---|---|---|---|---|---|---|---|
| Fluid | Water | A | B | C | D | E | F | G | H | I | J | Air⁽¹⁾ |
| NA | 1.2 | 1.2 | 1.2 | 1.2 | 1.2 | 1.2 | 1.2 | 1.2 | 1.2 | 1.2 | 1.2 | 0.35 |
| Δn | 0.0262 | 0.0100 | 0.0050 | 0.0020 | 0.0010 | 0.0005 | 0.0004 | 0.0003 | 0.0002 | 0.0001 | 0 | 0.3631 |
| RMS Primary | 0.0113 | 0.0103 | 0.0060 | 0.0026 | 0.0013 | 0.0007 | 0.0005 | 0.0004 | 0.0003 | 0.0001 | 0 | 0.0096 |
| SPHA (waves) | | | | | | | | | | | | |
| RMS High- | 0.0292 | 0.0105 | 0.0052 | 0.0020 | 0.0010 | 0.0005 | 0.0004 | 0.0003 | 0.0002 | 0.0001 | 0 | 0.0003 |
| order SPHA | | | | | | | | | | | | |
| (waves) | | | | | | | | | | | | |

As can be seen from table 1, in this example, diffraction limited performance can be achieved as long as the difference in refractive index between the buffer fluid and the immersion fluid is no more than 0.0003, meaning that immersion fluids G, H, I or J could be used, despite the fact only immersion fluid J had the same refractive index as the buffer fluid used in the system.

As another type of variation, while the above examples discussed a system where the objective was configured to compensate for expected aberration during top surface imaging and then the manipulable parameters were selected to allow the bottom surface to match, it is possible that an objective could be configured to compensate for expected aberration during bottom surface imaging, with manipulable parameters selected to match top surface imaging to this aberration. It is also possible that the above-described approaches could be applied to more than two surfaces, such as by selecting an immersion fluid and manipulating the position of the objective between imaging runs such that the difference between axial and marginal rays was the same for each surface. Other variations (e.g., setting a system's manipulable parameters to reduce aberration sufficiently to allow for a less complicated compensator to be used, rather than setting them to remove the need for a compensator entirely) are also possible, and will be immediately apparent to those of skill in the art in light of this disclosure. Accordingly, the above descriptions of approaches to addressing differences in aberration between surfaces, as well as the exemplary variations thereon, should be understood as being illustrative only, and should not be treated as limiting.

In particular embodiments, a system implemented based on this disclosure may utilize sequencing-by-synthesis (SBS). In SBS, one or more fluorescently labeled modified nucleotides are used to determine the sequence of nucleotides for nucleic acids present on the surface of a support structure such as a flow cell. Exemplary SBS systems and methods which can be utilized with the apparatus and methods set forth herein are described in U.S. Pat. No. 7,057,026; U.S. Patent Application Publication Nos. 2005/0100900, 2006/0188901, 2006/0240439, 2006/0281109, and 2007/0166705; and PCT Publication Nos. WO 05/065814, WO 06/064199, and WO 07/010251; each of which is incorporated herein by reference.

In particular uses of the apparatus and methods herein, flow cells containing arrayed nucleic acids are treated by several repeated cycles of an overall sequencing process. The nucleic acids are prepared such that they include an oligonucleotide primer adjacent to an unknown target sequence. To initiate the first SBS sequencing cycle, one or more differently labeled nucleotides and a DNA polymerase are flowed into the flow cell. Either a single nucleotide can be added at a time, or the nucleotides used in the sequencing procedure can be specially designed to possess a reversible termination property, thus allowing each cycle of the sequencing reaction to occur simultaneously in the presence of all four labeled nucleotides (A, C, T, G). Following nucleotide addition, the features on the surface can be imaged to determine the identity of the incorporated nucleotide (based on the labels on the nucleotides). Then, reagents can be added to the flow cell to remove the blocked 3′ terminus (if appropriate) and to remove labels from each incorporated base. Such cycles are then repeated and the sequence of each cluster is read over the multiple chemistry cycles. In further implementations, a fluorinated nucleotide or other detectable component may only temporarily associate with the unknown target sequence for imaging in a step separate from advancing to the next nucleotide.

Other sequencing methods that use cyclic reactions wherein each cycle includes steps of delivering one or more reagents to nucleic acids on a surface and imaging the surface bound nucleic acids can also be used such as pyrosequencing and sequencing by ligation. Useful pyrosequencing reactions are described, for example, in U.S. Pat. No. 7,244,559 and U.S. Patent Application Publication No. 2005/0191698, each of which is incorporated herein by reference. Sequencing by ligation reactions are described, for example, in Shendure et al. Science 309:1728-1732 (2005); and U.S. Pat. Nos. 5,599,675 and 5,750,341, each of which is incorporated herein by reference.

The methods and apparatus described herein are also useful for detection of features occurring on surfaces used in genotyping assays, expression analyses and other assays known in the art such as those described in U.S. Patent Application Publication Nos. 2003/0108900, US 2003/0215821, and US 2005/0181394, each of which is incorporated herein by reference.

While various implementations of the present disclosure have been described above, it should be understood that they have been presented by way of example only, and not of limitation. Likewise, the various diagrams may depict an example architectural or other configuration for the disclosure, which is done to aid in understanding the features and functionality that may be included in the disclosure. The disclosure is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the present disclosure. Also, a multitude of different constituent module names other than those depicted herein may be applied to the various partitions. Additionally, with regard to flow diagrams, operational descriptions and method claims, the order in which the acts are presented herein shall not mandate that various implementations be implemented to perform the recited functionality in the same order unless the context dictates otherwise.

1. A machine comprising:
a lens, the lens having a field of view and being immersed in a first fluid having a first refractive index;
a flow cell comprising first and second surfaces separated by a second fluid having a second refractive index; and
a controller, the controller to:
move the lens from a first position having a first distance to the flow cell to a second position having a second distance to the flow cell;
using the lens, capture light emitted from nucleic acids disposed on the first surface of the flow cell when the lens is separated from the flow cell by the first distance;
using the lens, capture light emitted from nucleic acids disposed on the second surface of the flow cell when the lens is separated from the flow cell by the second distance; and
determine a nucleic acid sequence for a biological sample based on the emitted light;
wherein an optical path difference between marginal light rays and axial light rays in the field of view of the lens when the lens is separated from the flow cell by the first distance is substantially equal to an optical path difference between marginal light rays and axial light rays in the field of view of the lens when the lens is separated from the flow cell by the second distance.

2. The machine of claim 1, wherein the first refractive index and the second refractive index are substantially equal.

3. The machine of claim 2, wherein:
the lens is comprised by optics adapted to capture light emitted from nucleic acid sequences disposed on the first surface of the flow cell, and to allow the biological sample to be imaged on a detector with diffraction limited imaging quality; and
the first refractive index and the second refractive index are substantially equal means spherical aberration caused by any difference between the first refractive index and the second refractive index is low enough not to prevent diffraction limited imaging of the biological sample based on:
light emitted from nucleic acid sequences disposed on the first surface of the flow cell; and
light emitted from nucleic acid sequences disposed on the second surface of the flow cell.

4. The machine of claim 2, wherein the first fluid and the second fluid are the same fluid.

5. The machine of claim 2, wherein the first fluid and the second fluid are different fluids.

6. The machine of claim 1, wherein the first surface of the flow cell is a top surface of the flow cell, and the second surface of the flow cell is a bottom surface of the flow cell.

7. The machine of claim 1, wherein:
the lens is comprised by optics adapted to capture light emitted from nucleic acid sequences disposed on the first surface of the flow cell, and to allow the biological sample to be imaged on a detector with diffraction limited imaging quality; and
the optical path difference between marginal light rays and axial light rays in the field of view of the lens when the lens is separated from the flow cell by the first distance is substantially equal to the optical path difference between marginal light rays and axial light rays in the field of view of the lens when the lens is separated from the flow cell by the second distance means spherical aberration is low enough not to prevent diffraction limited imaging of the biological sample based on:
light emitted from nucleic acid sequences disposed on the first surface of the flow cell; and
light emitted from nucleic acid sequences disposed on the second surface of the flow cell.

8. A method comprising:
capturing light emitted from nucleic acids disposed on a first surface of a flow cell using a lens which has a field of view and which is:
at a first distance from the flow cell; and
immersed in a first fluid having a first refractive index;
moving the lens to a position a second distance from the flow cell;
capturing light emitted from nucleic acids disposed on a second surface of the flow cell using the lens immersed in the first fluid having the first refractive index while the lens is at the second distance from the flow cell, wherein the first surface of the flow cell is separated from the second surface of the flow cell by a second fluid having a second refractive index; and
determining a nucleic acid sequence for a biological sample based on the light emitted from nucleic acids disposed on the first surface of the flow cell and the light emitted from the nucleic acids disposed on the second surface of the flow cell;
wherein an optical path difference between marginal light rays and axial light rays in the field of view of the lens when the lens is at the first distance from the flow cell is substantially equal to an optical path difference between marginal light rays and axial light rays in the field of view of the lens when the lens is at the second distance from the flow cell.

9. The method of claim 8, wherein the first refractive index and the second refractive index are substantially equal.

10. The method of claim 9, wherein:
the lens is comprised by optics adapted to capture light emitted from nucleic acid sequences disposed on the first surface of the flow cell, and to allow the biological sample to be imaged on a detector with diffraction limited imaging quality; and
the first refractive index and the second refractive index are substantially equal means spherical aberration caused by any difference between the first refractive index and the second refractive index is low enough not to prevent diffraction limited imaging of the biological sample based on:
light emitted from nucleic acid sequences disposed on the first surface of the flow cell; and
light emitted from nucleic acid sequences disposed on the second surface of the flow cell.

11. The method of claim 9, wherein the first fluid and the second fluid are the same fluid.

12. The method of claim 9, wherein the first fluid and the second fluid are different fluids.

13. The method of claim 8, wherein the first surface of the flow cell is a top surface of the flow cell, and the second surface of the flow cell is a bottom surface of the flow cell.

14. The method of claim 8, wherein:
the lens is comprised by optics adapted to capture light emitted from nucleic acid sequences disposed on the first surface of the flow cell, and to allow the biological sample to be imaged on a detector with diffraction limited imaging quality; and
the optical path difference between marginal light rays and axial light rays in the field of view of the lens when the lens at the first distance from the flow cell is substantially equal to the optical path difference between marginal light rays and axial light rays in the field of view of the lens when the lens at the second distance from the flow cell means spherical aberration is low enough not to prevent diffraction limited imaging of the biological sample based on:
light emitted from nucleic acid sequences disposed on the first surface of the flow cell; and
light emitted from nucleic acid sequences disposed on the second surface of the flow cell.

15. A non-transitory computer readable medium storing instructions to, when executed by a processor, cause a biological sample imaging system to perform acts comprising:
capture light emitted from nucleic acids disposed on a first surface of a flow cell using a lens at a first distance from the flow cell which has a field of view and which is immersed in a first fluid having a first refractive index;
move the lens to a position a second distance from the flow cell;
capture light emitted from nucleic acids disposed on a second surface of the flow cell using the lens immersed in the first fluid having the first refractive index while the lens is at the second distance from the flow cell, wherein the first surface of the flow cell is separated from the second surface of the flow cell by a second fluid having a second refractive index; and
determine a nucleic acid sequence for a biological sample based on the light emitted from nucleic acids disposed on the first surface of the flow cell and the light emitted from the nucleic acids disposed on the second surface of the flow cell;
wherein an optical path difference between marginal light rays and axial light rays in the field of view of the lens when the lens is at the first distance from the flow cell is substantially equal to an optical path difference between marginal light rays and axial light rays in the field of view of the lens when the lens is at the second distance from the flow cell.

16. The non-transitory computer readable medium of claim 15, wherein the first refractive index and the second refractive index are substantially equal.

17. The non-transitory computer readable medium of claim 16, wherein:
the lens is comprised by optics adapted to capture light emitted from nucleic acid sequences disposed on the first surface of the flow cell, and to allow the biological sample to be imaged on a detector with diffraction limited imaging quality; and
the first refractive index and the second refractive index are substantially equal means spherical aberration caused by any difference between the first refractive index and the second refractive index is low enough not to prevent diffraction limited imaging of the biological sample based on:
light emitted from nucleic acid sequences disposed on the first surface of the flow cell; and
light emitted from nucleic acid sequences disposed on the second surface of the flow cell.

18. The non-transitory computer readable medium of claim 15, wherein the first surface of the flow cell is a top surface of the flow cell, and the second surface of the flow cell is a bottom surface of the flow cell.

19. The non-transitory computer readable medium of claim 15, wherein:
the lens is comprised optics adapted to capture light emitted from nucleic acid sequences disposed on the first surface of the flow cell, and to allow the biological sample to be imaged on a detector with diffraction limited imaging quality; and
the optical path difference between marginal light rays and axial light rays in the field of view of the lens when the lens at the first distance from the flow cell is substantially equal to the optical path difference between marginal light rays and axial light rays in the field of view of the lens when the lens at the second distance from the flow cell means spherical aberration is low enough not to prevent diffraction limited imaging of the biological sample based on:
light emitted from nucleic acid sequences disposed on the first surface of the flow cell; and
light emitted from nucleic acid sequences disposed on the second surface of the flow cell.

20. The non-transitory computer readable medium of claim 15, wherein the first fluid and the second fluid are the same fluid.
